(19) United States
(12) Patent Application Publication
(10) Pub. No.: US 2023/0149697 A1
(43) Pub. Date: May 18, 2023

(54) VARIABLE STIFFNESS CANNULA

(71) Applicant: Boston Scientific Scimed Inc., Maple Grove, MN (US)

(72) Inventors: Brice Lee Shireman, Maple Grove, MN (US); Darrin Dale Beekman, Osseo, MN (US); Reggie Roth, Monticello, MN (US)

(21) Appl. No.: 17/989,005

(22) Filed: Nov. 17, 2022

Related U.S. Application Data
(60) Provisional application No. 63/280,209, filed on Nov. 17, 2021.

(51) Int. Cl. A61M 60/174; A61M 60/237

(52) U.S. Cl. CPC A61M 60/174; A61M 60/237; A61M 2205/0222; A61M 2205/0266; A61M 2207/10

(57) ABSTRACT

A percutaneous circulatory support device includes a housing, a cannula coupled to the housing, the cannula having a first portion and a second portion, and wherein the cannula includes at least one slot disposed along at least the first portion of the cannula, the at least one slot configured such that the first portion of the cannula is defined by a first stiffness and a second portion of the cannula is defined by a second stiffness, the first stiffness being different than the second stiffness.

### CROSS-REFERENCE TO RELATED APPLICATION

The present application claims priority to Provisional Application No. 63/280,209, filed Nov. 17, 2021, which is herein incorporated by reference in its entirety.

### TECHNICAL FIELD

The present disclosure relates to percutaneous circulatory support devices. More specifically, the disclosure relates to percutaneous circulatory support devices having a cannula.

### BACKGROUND

Circulatory support devices support the pumping action of the heart. These devices may be disposed through a valve opening such as, for example, an aortic valve. Blood flow through the circulatory support devices is an important factor when differentiating between different types of circulatory support devices. In some instances, a cannula may be used for providing blood flow through the circulatory support device. Proper positioning and stability of the cannula are important factors for retaining the proper blood flow and function of the circulatory support device.

### SUMMARY

In Example 1, a percutaneous circulatory support device includes a housing, a cannula coupled to the housing, the cannula having a first portion and a second portion. The cannula includes at least one slot disposed along at least the first portion of the cannula, the at least one slot configured such that the first portion of the cannula is defined by a first stiffness and a second portion of the cannula is defined by a second stiffness, the first stiffness being different than the second stiffness.

In Example 2, the percutaneous circulatory support device of Example 1 includes wherein the at least one slot is formed by laser cutting.

In Example 3, the percutaneous circulatory support device of Example 1, includes wherein the first portion is defined as a distal portion of the cannula and the second position is defined as a proximal portion of the cannula, and the first stiffness is less than the second stiffness.

In Example 4, the percutaneous circulatory support device of Example 1, further includes wherein the at least one slot includes a plurality of circular openings extending through the cannula.

In Example 5, the percutaneous circulatory support device of Example 1, further includes wherein the at least one slot includes a plurality of slots extending around the cannula.

In Example 6, the percutaneous circulatory support device of Example 1, further includes wherein the at least one slot defines a spiral slot extending around and along the cannula.

In Example 7, the percutaneous circulatory support device of Example 1, further includes wherein the cannula includes a coating configured to reduce the coefficient of friction of the cannula.

In Example 8, the percutaneous circulatory support device of Example 1, further includes wherein the cannula includes a shape set curved portion.

In Example 9, the percutaneous circulatory device of Example 3, further includes wherein the distal portion of the cannula includes an atraumatic tip element.

In Example 10, a percutaneous circulatory support device includes an impeller disposed within an impeller housing, the impeller being rotatable relative to the impeller housing to cause blood to flow through the impeller housing, and a motor configured to rotatably drive the impeller within the impeller housing. The device additionally includes a cannula coupled to the impeller housing, the cannula having a proximal portion, a distal portion, and an intermediate portion. The device further includes the cannula having at least one slot extending through the cannula and disposed along at least a first portion of the cannula, the at least one slot formed from laser cutting, and the first portion of the cannula body being defined by a first stiffness and a second portion of the cannula body being defined by a second stiffness that is different than the first stiffness.

In Example 11, the percutaneous circulatory support device of Example 10 further includes wherein the cannula comprises a curved portion, and wherein the curved portion defines a third portion having a third stiffness.

In Example 12, the percutaneous circulatory support device of Example 10 further includes wherein the distal portion of the cannula is operatively coupled to an atraumatic tip.

In Example 13, a method of forming a cannula of a percutaneous circulatory device, the percutaneous circulatory device including an impeller disposed within an impeller housing, the impeller being rotatable relative to the impeller housing to cause blood to flow through the percutaneous circulatory support device, wherein the cannula is configured to provide blood flow into the impeller housing, includes providing a cannula composed of a metallic material, the cannula having a distal portion, a proximal portion, an intermediate portion and a cannula lumen, the cannula lumen extending between the distal portion and the proximal portion. The method further includes shape setting the cannula such that the cannula tube comprises a curved portion, and laser cutting the cannula tube to form at least one slot within the tube such that the proximal portion has a first density of the at least one slot at a first portion of the cannula and a second density of the at least one slot at a second portion of the cannula, the first density being different than the second density.

In Example 14, the method of Example 13 further includes, wherein the at least one slot includes a plurality of openings extending circumferentially around the cannula.

In Example 15, the method of Example 13 further includes wherein the at least one slot includes at least one spiral cut extending along the cannula.

In Example 16, a percutaneous circulatory support device includes an impeller disposed within an impeller housing, the impeller being rotatable relative to the impeller housing to cause blood to flow through the impeller housing and a cannula coupled to the impeller housing, the cannula having a first portion and a second portion. The percutaneous circulatory support device additionally includes wherein the cannula includes at least one slot disposed along at least the first portion of the cannula, the at least one slot configured such that the first portion of the cannula body is defined by a first stiffness and a second portion of the cannula is defined by a second stiffness, the first stiffness being different than the second stiffness.

In Example 17, the device of Example 16 further includes wherein the first portion is defined as a distal portion and the second portion is defined as a proximal portion, and the first stiffness is less than the second stiffness.

In Example 18, the device of Example 16 further includes wherein the at least one slot includes a plurality of openings, and wherein the first portion has a reduced density of the plurality of openings relative to a density of the plurality of openings at the second portion.

In Example 19, the device of Example 16 further includes wherein the at least one slot includes a plurality of circular openings extending through the cannula.

In Example 20, the device of Example 16 further includes wherein the at least one slot includes a plurality of slots extending around the cannula.

In Example 21, the device of Example 16 further includes wherein the at least one slot defines a spiral slot extending around and along the cannula.

In Example 22, the device of Example 16 further includes wherein the cannula body comprises a shape set curved portion.

In Example 23, the device of Example 16 further includes wherein the distal portion of the cannula comprises an atraumatic tip element.

In Example 24, the device of Example 16 further includes wherein the cannula includes a coating configured to reduce the coefficient of friction of the cannula.

In Example 25, the device of Example 16 further includes wherein the cannula is composed of one of nitinol, stainless steel, Inconel and MP35N.

In Example 26, a percutaneous circulatory support device includes an impeller disposed within an impeller housing, the impeller being rotatable relative to the impeller housing to cause blood to flow through the impeller housing and a motor configured to rotatably drive the impeller within the impeller housing. The percutaneous support device further includes a cannula coupled to the impeller housing, the cannula having a proximal portion, a distal portion, and an intermediate portion, and wherein the cannula includes at least one slot extending through the cannula body disposed along at least the distal portion of the cannula, wherein the at least one slot is formed from laser cutting, the distal portion of the cannula being defined by a first stiffness and the proximal portion of the cannula being defined by a second stiffness that is different than the first stiffness.

In Example 27, the device of Example 26 further includes wherein the cannula comprises a curved portion in the intermediate portion, and wherein the intermediate portion defines a third portion having a third stiffness.

In Example 28, the device of Example 26 further includes wherein the third stiffness is different than the first stiffness and the second stiffness.

In Example 29, the device of Example 26 further includes wherein the distal portion of the cannula is operatively coupled to an atraumatic tip.

In Example 30, a method of forming a cannula of a percutaneous circulatory device, the percutaneous circulatory device including an impeller disposed within an impeller housing, the impeller being rotatable relative to the impeller housing to cause blood to flow through the impeller housing, wherein the cannula is configured to provide blood flow into the impeller, includes providing a cannula composed of a metallic material, the cannula having a distal portion, a proximal portion, and a cannula lumen extending between the proximal portion and the distal portion. The method further includes shape setting the cannula such that the cannula tube includes a curved portion and laser cutting the cannula tube to form at least one slot within the cannula such that the proximal portion has a first density of the at least one slot at a first portion of the cannula and a second density of the at least one slot at a second portion of the cannula, the first density being different than the second density.

In Example 31, the method of Example 31 further includes attaching a tip element to the distal portion of the cannula.

In Example 32, the method of Example 30 further includes wherein the metallic material is one of nitinol, stainless shell, Inconel, and MP35N.

In Example 33, the method of Example 30 further includes wherein the at least one slot includes a plurality of openings extending circumferentially around the cannula.

In Example 34, the method of Example 30 further includes wherein the at least one slot includes at least one spiral cut extending along the cannula.

In Example 35, the method of Example 30 further includes an intermediate portion extending between the proximal portion and the distal portion, and wherein the intermediate portion includes the curved portion.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a conceptual diagram of a circulatory support device including a cannula and a connector, in accordance with embodiments of the subject matter disclosed herein.

FIG. 2 is a side sectional view of several components of an illustrative percutaneous circulatory support device, in accordance with embodiments of the subject matter disclosed herein.

FIG. 3 is a side view of a cannula of the illustrative percutaneous circulatory support device of FIG. 2, in accordance with embodiments of the subject matter disclosed herein.

FIG. 4 is a side view of a cannula of the illustrative percutaneous circulatory support device of FIG. 1, in accordance with embodiments of the subject matter disclosed herein.

FIG. 5 is a side view of a cannula of the illustrative percutaneous circulatory support device of FIG. 1, in accordance with embodiments of the subject matter disclosed herein.

FIG. 6 illustrates various embodiments of a plurality of openings of a cannula, in accordance with embodiments of the subject matter disclosed herein.

### DETAILED DESCRIPTION

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features.

Embodiments disclosed herein include circulatory support devices that have an increased flow capability in comparison to conventional embodiments.

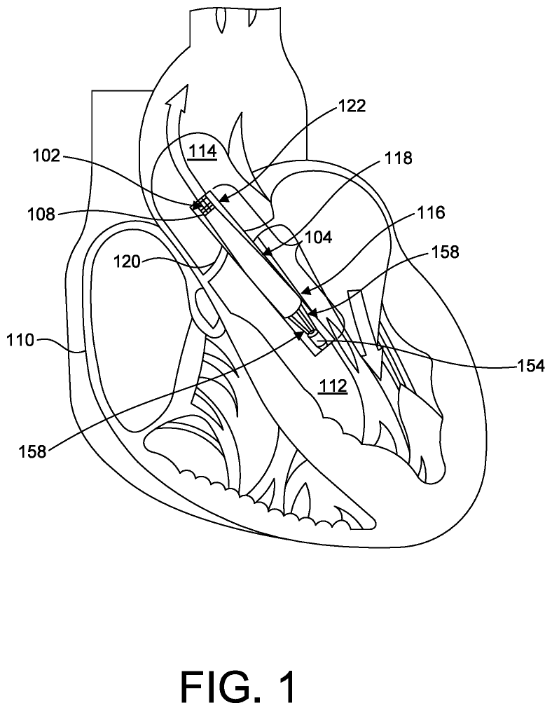

FIG. 1 depicts a conceptual diagram of a circulatory support device 102, including a cannula 104 and a connector 108, in accordance with embodiments of the subject matter disclosed herein. The circulatory support device 102 is shown arranged within a heart 110. According to embodiments, the circulatory support device 102 (also referred to herein, interchangeably, as a “blood pump”) is coupled to the cannula 104 by the connector 108. The circulator support device 102 is configured to pump blood from the subject's left ventricle 112 into the subject's aorta 114. In embodiments, the circulatory support device 102 may be used to treat cardiogenic shock and other heart failure modalities.

In embodiments, a distal portion 116 of the cannula 104 is arranged in the left ventricle 112. An intermediate portion 118 of the cannula 104 extends through the aortic valve 120 so that a proximal portion 122 of the cannula 104 extends into the aorta 114. In embodiments, the proximal portion 122 of the cannula 104 is coupled to the connector 108 and the connector 108 is coupled to the circulatory support device 102. In other embodiments, the cannula 104 is coupled to the circulatory support device without the use of a connector. During operation, the circulatory support device 102 draws blood from the left ventricle 112, through the cannula 104 of the circulatory support device 102 and is released into the aorta 114. Additionally, or alternatively, the circulatory support device 102 may be used to facilitate pumping blood from some other aspect of the subject's vasculature into an adjacent portion of the vasculature.

FIG. 1 is not intended to suggest any limitation as to the scope of use or functionality of embodiments of the present disclosure. FIG. 1 also should not be interpreted as having any dependency or requirement related to any single component or combination of components illustrated therein. Additionally, various components depicted in FIG. 1 may be, in embodiments, integrated with various ones of the other components depicted therein (and/or components not illustrated), all of which are considered to be within the ambit of the present disclosure.

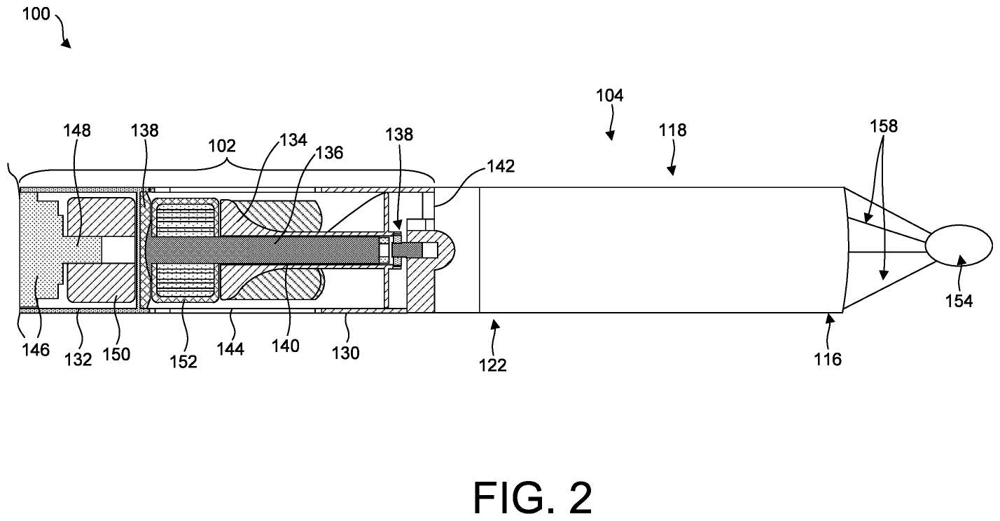

FIG. 2 depicts a partial side sectional view of the circulatory support device 102 depicted in FIG. 1 and the cannula 104, in accordance with embodiments of the subject matter disclosed herein. As previously disclosed with reference to FIG. 1, the cannula 104 may include a proximal portion 122, an intermediate portion 118, and a distal portion 116. The proximal portion 122 is positioned coupled to the connector 108 which is then operatively coupled to the remainder of the circulatory support device 102, the components of which will be described further herein.

Further, as illustrated, the cannula 104 may comprise a tip element 154 attached to the distal portion 116 of the cannula 104. As illustrated, the tip element 154 is a spherical element that is coupled to the distal portion 116 through the use of a plurality of wires 158. Specifically, the plurality of wires 158 are coupled to the distal portion 116 of the cannula 104 and coupled directly to the tip element 154. The tip element 154 may prevent suction of tissue into the cannula 104 by spacing the distal portion 116 away from tissue. While illustrated as a sphere, the tip element 154 may be any variety of shapes, for example a rectangle or a cylinder. Additionally, or alternatively, the tip element 154 may be radiopaque to help determine proper positioning of the cannula 104 during or after delivery. Further, the spaces between the plurality of wires 158 may act as an inlet for the blood to enter the cannula 104. In other embodiments, an inlet may be defined by other features, such as a housing coupling the intermediate portion 118 of the cannula 104 to the tip element 154. Additionally, or alternatively, tip element 154 acts as an atraumatic tip element configured to protect the patient's vasculature or the tissue of the heart 110, including valvular tissue, by eliminating harsh edges or surfaces that could come into contact with the surrounding vasculature or tissue as the cannula 104 is navigated to, into, or through the heart 110. For example, the tip element 154 may include a solder ring, a balloon and/or a silicone ring to serve as an atraumatic tip element. Further, in other embodiments, an elongated tip, for example taking shape similar to a guidewire, is used as the tip element 154. In some embodiments, no tip element is incorporated with the cannula 104 at all.

With continued reference to FIG. 2, the circulatory support device 102 generally includes an impeller housing 130 and a motor housing 132. In some embodiments, the impeller housing 130 and the motor housing 132 may be integrally or monolithically constructed. In other embodiments, the impeller housing 130 and the motor housing 132 may be separate components configured to be removably or permanently coupled.

The impeller housing 130 carries an impeller assembly 134 therein. The impeller assembly 134 includes an impeller shaft 136 that is rotatably supported by at least one bearing, such as a bearing 138. The impeller assembly 134 also includes an impeller 140 that rotates relative to the impeller housing 130 to drive blood through the device 102. More specifically, the impeller 140 causes blood to flow from a blood inlet 142 formed on the impeller housing 130, through the impeller housing 130, and out of a blood outlet 144 formed on the impeller housing 130. In some embodiments and as illustrated, the impeller shaft 136 and the impeller 140 may be separate components, and in other embodiments the impeller shaft 136 and the impeller 140 may be integrated. In some embodiment and as illustrated, the inlet 142 and/or the outlet 144 may each include multiple apertures. In other embodiments, the inlet 142 and/or the outlet 144 may each include a single aperture. In some embodiments and as illustrated, the inlet 142 may be formed on an end portion of the impeller housing 130 and the outlet 144 may be formed on a side portion of the impeller housing 130. In other embodiments, the inlet 142 and/or the outlet 144 may be formed on other portions of the impeller housing 130. As illustrated and previously described, the impeller housing 130 couples to the cannula 104 such that the cannula 104 receives and delivers blood to the blood inlet 142.

With continued reference to FIG. 2, the motor housing 132 carries a motor 146, and the motor 146 is configured to rotatably drive the impeller 140 relative to the impeller housing 130. In the illustrated embodiment, the motor 146 rotates a drive shaft 148, which is coupled to a driving magnet 150. Rotation of the driving magnet 150 causes rotation of a driven magnet 152. The driven magnet 152 is connected to and rotates together with the impeller assembly 134. More specifically, in embodiments incorporating the impeller shaft 136, the impeller shaft 136 and the impeller 140 are configured to rotate with the driven magnet 152. In other embodiments, the motor 146 may couple to the impeller assembly 134 via other components.

In some embodiments, a controller (not shown) may be operably coupled to the motor 146 and configured to control the motor 146. In some embodiments, the controller may be disposed within the motor housing 132. In other embodiments, the controller may be disposed outside of the motor housing 132 (for example, in a catheter handle, an independent housing, etc.). However, the above described embodiment of the circulatory support device 102 is not meant to be limiting and the cannula 104, and any variations of the cannula described herein with reference to FIGS. 1-6, may be used with variations of other circulatory support devices. Even further, the cannula 104 described herein may be used with various other percutaneous devices. The cannula 104, and various embodiments thereof, will be described further herein.

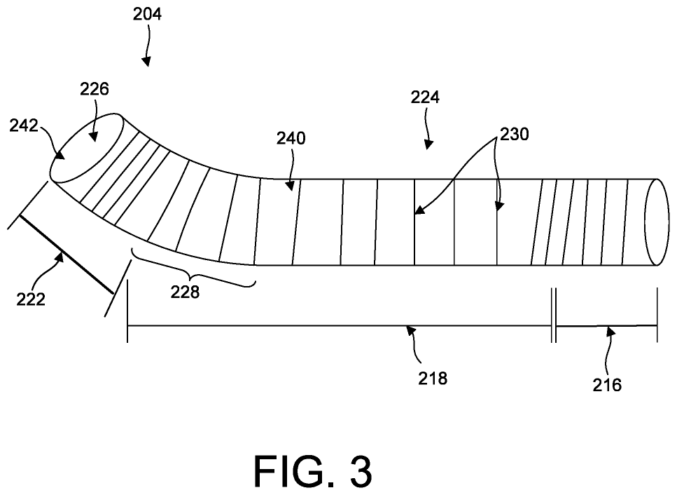

FIG. 3 illustrates an embodiment of a cannula 204 that may be used in combination with the circulatory support device of FIG. 1. Similar to the cannula 104 of FIG. 1, the cannula 204 comprises a proximal portion 222, an intermediate portion 218, and a distal portion 216. Additionally, the cannula 204 comprises a curved portion 228 within the intermediate portion 218. In some embodiments, the cannula 204 includes a plurality of curved portions at different positions along the cannula 204. The cannula 204 comprises a cannula body 224 and a lumen 226 extending between the proximal portion 222 and the distal portion 216. The lumen 226 is configured for the passage of blood into the cannula 204 and into the blood inlet 142 (FIG. 2) of the device 102 (FIG. 1). As shown, the cannula lumen 226 is generally cylindrical in shape, however, various other shapes and configurations may be incorporated. The cannula 204 may be formed from a variety of materials. For example, the cannula 204 may be composed of materials including, but not limited to, nitinol, stainless steel, Inconel, and MP35N. The use of the above present materials, or various other appliable materials, allows for the cannula 204 to be shape set into a desired shape or configuration, as well as the ability for the cannula 204 to be laser cut or otherwise altered to have slots. For example, the cannula 204 may be formed by cutting slots into a hypo tube formed of any one of the above materials, as will be described further herein.

As shown in FIG. 3, the cannula 204 includes the curved portion 228 that is positioned adjacent the proximal portion 222 of the cannula 204 and within the intermediate portion 218 of the cannula 204. However, the positioning of the curved portion 228 may vary, for example it may be adjacent the distal portion 216 of the cannula 204. The curved portion 228 may be formed by shape setting the cannula 204, for example with a heat set. The configuration of the curved portion 228 of FIG. 3 is not meant to be limiting, and may comprise a radius of curvature that is greater than, equal to, or less than the radius of curvature shown in FIG. 3. In various embodiments, the curved portion 228 may provide at least the advantage of optimized shaping of the cannula 204 relative to the native anatomy of the heart 110 (FIG. 1) when in use with the device 102 (FIG. 1). For example, when the cannula 204 is positioned within the heart 110, the curved portion 228 may be positioned just within the aorta 114 to provide a better positioning of the cannula 204 within the heart 110, for example with respect to the aortic valve and ventricular walls. In other embodiments, the curved portion 228 may be positioned just above or below the aorta 114. An additional advantage of the curved portion 228 may be allowing for an easier delivery of the cannula 204 into the heart 110, as the curved portion 228 may align with the curvature of the chambers in the heart 110.

The shape of the cannula 204 may also be manipulated by hand, for example by a physician adjusting the shape of the cannula 204 prior to insertion into a patient. For example, the curved portion 228 may be formed by a physician prior to the cannula 204 being inserted into a patient's vasculature.

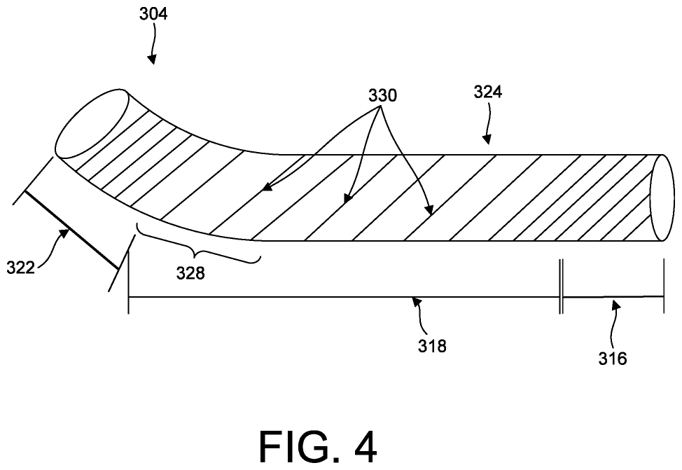

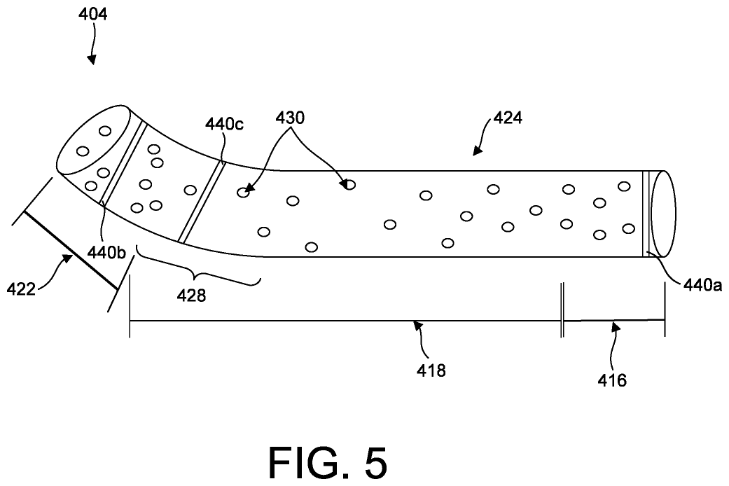

As previously mentioned, an additional advantage of the cannula 204 as disclosed herein, is the ability for slots to be cut into the cannula 204, for example by laser cutting the cannula 204. For example, as illustrated in FIG. 3, the cannula 204 comprises at least one slot disposed along the cannula 204. In the embodiment of FIG. 3, the at least one slot comprises a plurality of slots 230 extending circumferentially around the cannula 204. As will be described further with reference to FIGS. 4-5, the plurality of slots 230 may take various other forms or shapes. With continued reference to FIG. 3, each of the plurality of slots 230 extends through the cannula 204 such that it forms an opening extending from an exterior of the cannula 204 to the cannula lumen 226. However, in various other embodiments, the plurality of slots 230 may extend through only a portion of the cannula 204 such that the plurality of slots 230 extend a depth that is less than a thickness of the cannula 204. Stated differently, in some embodiments, the slots 230 may not form apertures in the cannula 204, but instead form areas of reduced thickness of the cannula 204.

The distribution of the plurality of slots 230 may be varied as well. For example, as illustrated in the embodiment of FIG. 3, the plurality of slots 230 have a higher density towards the proximal portion 222 and the distal portion 216 relative to the density of the plurality of slots 230 at the curved portion 228 and the intermediate portion 218 of the cannula 204. Additionally, the density of the plurality of slots 230 may be higher at the distal portion 216 than at the proximal portion 222. The density may be defined as the amount of open surface area within a given section of the cannula 204 relative to the amount of cannula material, for example nitinol, within the same given section of the cannula 204.

One advantage of the incorporation of the plurality of slots 230, and particularly with varying density along the cannula 204, allows for the cannula 204 to have a varying stiffness and/or varying amounts of flexibility throughout the cannula 204 while maintaining a constant wall thickness. For example, the distal portion 216 may be formed with a higher plurality of slots 230 in order to increase the flexibility at the distal portion 216 of the cannula 204. In this way, the cannula 204 is still capable of flexibility and a range of motion at the distal portion 216. Further, the cannula 204 may have a lower density of the plurality of slots 230 towards the curved portion 228 to increase the stiffness of the curved portion of the cannula 204. This may provide the advantage of increasing the stability of the curved portion 228 when it is positioned at the aorta 114 (FIG. 1) and increasing the trackability of the cannula 204. Further, in various embodiments, the plurality of slots 230 may have a higher density at the proximal portion 222 to increase the flexibility relative to the flexibility of the curved portion 228. In other embodiments, the plurality of slots 230 may have a lower density in the proximal portion 222 than the curved portion 228 to increase the flexibility of the curved portion 228 relative to the proximal portion 222. In this way, the stiffness of the various portions of the cannula 204 may easily be varied based on the amount of the plurality of slots 230 incorporated. In additional embodiments, the arclength of the material between the plurality of slots 230 of the cannula 204 may contribute to the varying stiffness values along the cannula 204 as well. In some embodiments, the stiffness at the proximal portion 222 is adjusted to match the stiffness of the component that the proximal portion 222 is attached to in assembly, such that there is a smooth transition between the stiffness of the cannula 204 and the adjacent component, for example, the connector 108 (FIG. 1). The above described relative stiffnesses are not meant to be limiting, and the at least one slot of the cannula 204 may be varied and distributed to best optimize the trackability and flexibility of the cannula 204. For example, the cannula 204 could comprise a continuously changing stiffness profile based on the configuration of the plurality of slots 230 to optimize the stiffness transition throughout the cannula 204. For example, the continuously changing stiffness profile could be defined by linear, parabolic, and/or polynomial curves to optimize the stiffness transitions.

Additionally, between each portion of the cannula 204 that defines varying stiffness values, the plurality of slots 230 may be configured to provide gradual increases (or decreases) in the stiffnesses. Incorporating gradual transition between the varying stiffness values may increase the stability of the cannula 204 as opposed to if abrupt changes in the stiffness were present. Varying the stiffness and/or varying the amounts of flexibility throughout the cannula 204 may also permit manipulation of the cannula 204 by the physician before or after insertion into the vasculature, as described above. For example, the pattern of the slots 230 may be designed to control the bending amount or direction of the cannula 204.

An additional advantage of using the plurality of slots 230 is the ability to optimize the insertion of the cannula 204 through the vasculature and into the heart 110 (FIG. 1). In particular, cannula 204 allows for delivery of a circulatory support device without the use of a guidewire. For example, the stiffness along the cannula 204 can be optimized such that the cannula 204 is flexible enough to be navigated and moved into the target position without damaging the tissue, yet stable enough to be passed through the patient's vasculature and the heart 110 and positioned properly and held in position within the heart 110. In other words, the cannula 204 needs to be flexible enough to maneuver through the curvature of the vascular system, but stable enough not to fold on itself or collapse while passing through the vascular system and into the heart. Thus, while a guidewire (not shown) may be incorporated to deliver the circulatory support device 102 to the target position, the optimized stiffness of the cannula 204 through incorporation of the plurality of slots 230 allows the cannula 204 to be delivered through the heart 110 without the use of a guidewire.

An additional feature that may be incorporated into the cannula 204 is a surface coating 240 disposed around the entirety of the cannula 204. In some embodiments, the surface coating 240 is a silicone, PET, or other biocompatible and/or hydrophobic polymers such as polyether block amide, polytetrafluoroethylene, fluorinated ethylene propylene, or polyurethane. In some embodiments, the surface coating 240 can be hydrophilic. The surface coating 240 may be applied via a dip coating, a spray coating, molding, heat shrink, or polymer reflow process among other methods. The surface coating 240 may be attached to the cannula body 224, and thus the cannula 204. The surface coating 240 may be provided for optimizing the cannula 204 for trackability and biocompatibility. For example, the surface coating 240 may reduce the coefficient of friction of the cannula 204 to increase the ease with which the cannula 204 is delivered into the heart 110. Additionally, the use of the surface coating 240 increases the biocompatibility of the cannula 204 to avoid undesired reactions between the cannula 204 and tissue of the vasculature. Further, the surface coating 240 may be applied such that the entirety of the outer surface and the inner surface of the cannula 204 is covered. In the embodiments wherein the plurality of slots 230 are a plurality of apertures extending entirely through the cannula 204, the surface coating 240 creates a seal over the plurality of slots 230 such that the cannula 204 is a sealed tube and does not allow for fluid or blood flow through the plurality of slots 230. Even further, the cannula 204 may comprise an additional surface coating 242 on an inner surface the cannula 204. Similar to the surface coating 240 as described with reference to FIG. 2, the surface coating 240 may be a silicone, PET or other polymer coating. The surface coatings 240, 242 may be applied to the embodiments of any one of FIGS. 1-5 herein, and are not limited to the use with the cannula 204 of FIG. 3.

FIG. 4 illustrates an additional embodiment of a cannula of the device 102 as described previously with reference to FIG. 1. Specifically, FIG. 4 is a side view of a cannula 304. The cannula 304 may be largely similar to the cannula 204 as described with reference to FIG. 3. For example, the cannula 304 comprises a distal portion 316, a curved portion 328 within an intermediate portion 318, and a proximal portion 322. The cannula 304 additionally comprises a cannula body 324 extending between the distal portion 316 and the proximal portion 322. However, the cannula 304 differs from cannula 204 in that the cannula 304 includes at least one slot that is different than the at least one slot 230 (FIG. 3). Specifically, the at least one slot includes a spiral cut 330 that extends around the cannula 304. As such, the cannula 304 comprises the spiral cut 330 extending at a diagonal and extending between the distal portion 316 and the proximal portion 322. The density of the spiral cut 330 along the cannula 304 may vary in density based on a distance between the circumferential segments of the spiral cut 330. In this way, the stiffness values of the cannula 304 can vary along the cannula 304 for providing the optimized delivery and anchoring of the cannula 304.

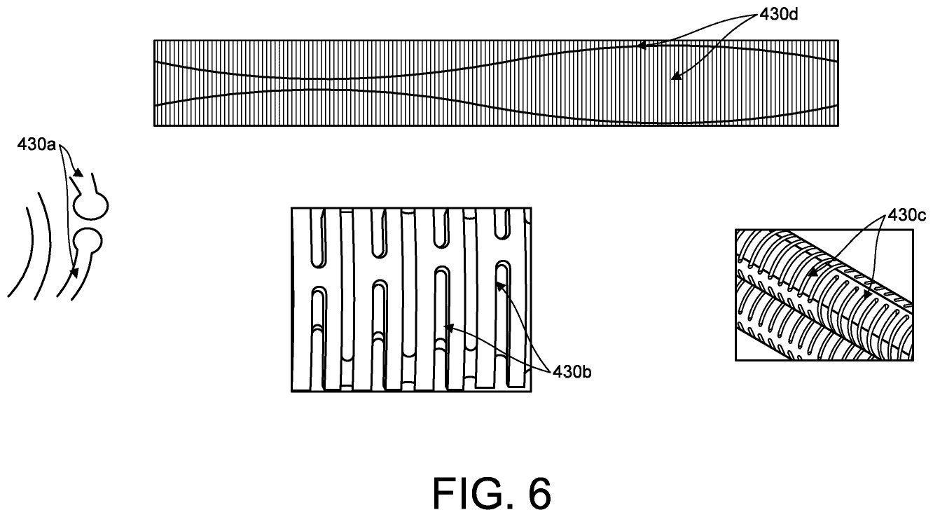

While the at least one slot described with reference to FIGS. 3 and 4 are illustrated generally as slots or thin cuts through the cannula 204, 304, at least one opening of a cannula may vary further in shape or configuration. For example, FIG. 5 illustrates an additional embodiment of a cannula 404. Similar to described herein, the cannula 404 includes a distal portion 416, a curved portion 428 within an intermediate portion 418, and a proximal portion 422. The cannula 404 comprises a cannula body 424 extending between the distal portion 416 and the proximal portion 422. Further, the cannula 404 comprises at least one slot. In this embodiment, the at least one slot comprises a plurality of openings 430 that are illustrated as generally circular holes extending through and along the cannula 404. However, the plurality of openings 430 may take on any desired shape or pattern. For example, the shape of the plurality of openings 430 may be shaped as a circle, oval, square, rectangle, or any other shape desired. For example, the openings 430 may have a shape that is a combination of the above, for example a dog bone shape defined by a cylinder joined with a circle at each end. Other examples of shapes and/or patterns that may define the plurality of openings 430 are included in FIG. 6. For example, FIG. 6 illustrates a plurality of openings 430a having a general dog bone shape. Additionally, FIG. 6 illustrates a plurality of openings 430b having generally rectangular openings disposed along the cannula 404 (FIG. 5) with a pattern. Further, FIG. 6 illustrates an additional configuration of the plurality of openings, illustratively a plurality of openings 430c that are generally rectangular or ovular slots that extend adjacent one another along the cannula 404. Further, the plurality of openings 430d illustrate a pattern of both circumferentially extending slots and longitudinally extending slots to form a pattern of the plurality of openings 430d within the cannula 404. However, the various shapes and patterns illustrated in FIG. 6 are not meant to be limiting, and various other shapes and/or patterns of the plurality of openings 430 may be incorporated. The plurality of openings 430 function similarly to the at least one slot 230 as described with reference to FIG. 3 and the spiral cut 330 as described with reference to FIG. 4, wherein the positioning and density of the openings 430 along the cannula 404 allows for a varied stiffness along the cannula 404.

While the following description is made with reference to the cannula 404 illustrated in FIG. 5, the following description applies to cannulas 104, 204, and 304 as described with reference to FIGS. 1-4. The plurality of openings 430 may be formed through laser cutting the cannula 404 or otherwise machining the cannula 404. In addition, in the embodiments wherein the cannula 404 is laser cut, additional surface features may be incorporated onto the cannula 404. For example, as illustrated in FIG. 5 the cannula 404 additionally includes surface features 440. Specifically, surface features 440 may be echogenic surface features 440 that are formed directly onto the cannula 404. This may be particularly advantageous in that after the cannula 404 and the circulatory support device 102 (FIG. 1) are positioned in the heart 110 (FIG. 1), a physician may conduct an imaging test, for example an ultrasound, of the heart 110 of the patient that reveals the positioning of the cannula 404 and the circulatory support device 102. The surface features 440 may comprise at least one surface feature 440, however various other amounts of surface features or markings can be imagined. For example, the cannula 404 can comprise two, three, four, five, or more surface features for indicating the position. For example, the cannula 404 may have a first surface feature 440a positioned at the distal portion 416 of the cannula 404 and/or a second surface feature 440b at the proximal portion 422 of the cannula 404. This may indicate to the physician the physical bounds of the cannula 404 once positioned. Further, the surface features 440 may include a third surface feature 440c positioned at a curved portion 428 of the cannula 404. As such, in embodiments wherein the curved portion 428 is positioned at or approximately at the aorta 114 (FIG. 1) of the heart 110 (FIG. 1), the physician can be indicated when the curved portion 428 is positioned at the aorta 114 (FIG. 1). The surface features 440 may comprise at least one, all, or any combination of the described surface features 440.

The described features of the cannulas 104, 204, and 404 are not meant to be limited to their embodiments. For example, various features as described with reference to cannula 204 may be used in combination with the cannula 404 or the cannula 104. For example, the tip element 154 may be incorporated into cannula 204 and/or cannula 304 and/or cannula 404 as well. Further, the surface coating 240 as described with reference to the cannula 204 may be used in combination with the cannulas 104, 304, and/or 404 as described with reference to FIGS. 1-2 and 4-5. Further, the surface features 440 as described with reference to the cannula 404 of FIG. 5 may be used in combination with the cannulas 104, 204, 304 as described with reference to FIGS. 1-4.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A percutaneous circulatory support device, comprising:
an impeller disposed within an impeller housing, the impeller being rotatable relative to the impeller housing to cause blood to flow through the impeller housing;
a cannula coupled to the impeller housing, the cannula having a first portion and a second portion; and
wherein the cannula includes at least one slot disposed along at least a first portion of the cannula, the at least one slot configured such that the first portion of the cannula is defined by a first stiffness and a second portion of the cannula is defined by a second stiffness, the first stiffness being different than the second stiffness.

2. The percutaneous circulatory support device of claim 1, wherein the first portion is defined as a distal portion of the cannula and the second portion is defined as a proximal portion of the cannula, and the first stiffness is less than the second stiffness.

3. The percutaneous circulatory support device of claim 1, wherein the at least one slot includes a plurality of openings, and wherein the first portion of the cannula has a reduced density of the plurality of openings relative to a density of a plurality of openings at the second portion of the cannula.

4. The percutaneous circulatory support device of claim 1, wherein the at least one slot includes a plurality of circular openings extending through the cannula.

5. The percutaneous circulatory support device of claim 1, wherein the at least one slot includes a plurality of slots extending around the cannula.

6. The percutaneous circulatory support device of claim 1, wherein the at least one slot defines a spiral slot extending around and along the cannula.

7. The percutaneous circulatory support device of claim 1, wherein the cannula comprises a shape set curved portion.

8. The percutaneous circulatory support device of claim 2, wherein the distal portion of the cannula comprises an atraumatic tip element.

9. The percutaneous circulatory support device of claim 1, wherein the cannula includes a coating configured to reduce the coefficient of friction of the cannula and create a seal over the at least one slot.

10. The percutaneous circulatory support device of claim 1, wherein the cannula is composed of one of nitinol, stainless steel, Inconel and MP35N.

11. A percutaneous circulatory support device, the device comprising:
a percutaneous circulatory support device, the device including,
an impeller disposed within an impeller housing, the impeller being rotatable relative to the impeller housing to cause blood to flow through the impeller housing, and
a motor configured to rotatably drive the impeller within the impeller housing; and
a cannula coupled to the impeller housing, the cannula having a proximal portion, a distal portion, and an intermediate portion; and
wherein the cannula includes at least one slot extending through the cannula and disposed along at least distal portion of the cannula, wherein the at least one slot is formed from laser cutting, the distal portion of the cannula being defined by a first stiffness, and the proximal portion of the cannula being defined by a second stiffness that is different than the first stiffness.

12. The percutaneous circulatory support device of claim 11, wherein the cannula comprises a curved portion in the intermediate portion, and wherein the intermediate portion defines a third portion having a third stiffness.

13. The percutaneous circulatory support device of claim 11, wherein the third stiffness is different than the first stiffness and the second stiffness.

14. The percutaneous circulatory support device of claim 11, wherein the distal portion of the cannula is operatively coupled to an atraumatic tip.

15. A method of forming a cannula for use with a percutaneous circulatory device, the percutaneous circulatory device including an impeller disposed within an impeller housing, the impeller being rotatable relative to the impeller housing to cause blood to flow through the impeller housing, wherein the cannula is configured to provide blood flow into the impeller, the method comprising:
providing a cannula composed of a metallic material, the cannula having a distal portion, a proximal portion, and a cannula lumen extending between the distal portion and the proximal portion;
shape setting the cannula such that the cannula comprises a curved portion; and
laser cutting the cannula to form at least one slot within the cannula such that the proximal portion has a first density of the at least one slot at a first portion of the cannula and a second density of the at least one slot at a second portion of the cannula, the first density being different than the second density.

16. The method of claim 15, further comprising attaching a tip element to the distal portion of the cannula.

17. The method of claim 15, wherein the metallic material is one of nitinol, stainless shell, Inconel and MP35N.

18. The method of claim 15, wherein the at least one slot includes a plurality of openings extending circumferentially around the cannula.

19. The method of claim 15, wherein the at least one slot includes at least one spiral cut extending along the cannula.

20. The method of claim 15, wherein the cannula further includes an intermediate portion extending between the proximal portion and the distal portion, and wherein the intermediate portion includes the curved portion.
